(19) United States
(12) Patent Application Publication
(10) Pub. No.: US 2026/0199251 A1
(43) Pub. Date: Jul. 16, 2026

(54) SOFT GELATIN CAPSULE AND PROCESS FOR PRODUCTION

(71) Applicant: Gelita AG, Eberbach (DE)

(72) Inventors: Ulrich MACH, Ulm (DE); Holger BECKER, Eberbach (DE); Martin MÜLLER, Oberzent (DE); Michelle MONTGOMERY, Jefferson, SD (US); Eric YEZDIMER, Sioux City, IA (US); Stephan HAUSMANNS, Heidelberg (DE); Denise REIS, Sao Paulo (BR)

(21) Appl. No.: 19/468,074

(22) Filed: Feb. 3, 2026

Related U.S. Application Data
(63) Continuation of application No. PCT/EP2024/072353, filed on Aug. 7, 2024.

(30) Foreign Application Priority Data
Aug. 8, 2023 (DE) 102023121056.4

(51) Int. Cl. A61K 9/48; C07K 14/78; C12N 9/50

(52) U.S. Cl. CPC A61K 9/4825; A61K 9/4833; C07K 14/78; C12N 9/50

(57) ABSTRACT

The present invention relates to a soft gelatin capsule comprising a capsule shell and a filling encased by the capsule shell. The capsule shell comprises a low-viscosity gelatin having a viscosity of about 1.5 to about 2.4 mPa·s and a gel strength of about 150 to about 280 g Bloom, preferably of about 180 to about 250 g Bloom, in each case measured in a 6.67% gelatin solution at 60° C. according to the GME monograph “Standardised Methods for the Testing of Edible Gelatin.”

### CROSS-REFERENCE TO RELATED APPLICATIONS

This patent application is a continuation of International Patent Application No. PCT/EP2024/072353, filed Aug. 7, 2024, which claims priority to German Patent Application No. 10 2023 121 056.4, filed Aug. 8, 2023, each of which is hereby incorporated by reference in its entirety.

### BACKGROUND OF THE INVENTION

Soft gelatin capsules have long been a well-known dosage form for pharmaceutical products and dietary supplements. They are particularly suitable also for encapsulating liquid or paste-like fillings, which enable rapid absorption of the active substances contained therein after the capsule shell has dissolved in the digestive tract. In addition to solutions or emulsions of active substances, hydrophobic liquids such as oils can also be administered using soft gelatin capsules.

Soft gelatin capsules are typically produced using the rotary die process. In this process, the surfaces of two gel ribbons comprising gelatin, plasticizer, and water are first melted superficially as they pass a filling wedge, pressed against each other, and thereby bonded together. Due to thermal equilibrium with the environment, the melt zones solidify quickly, resulting in the formation of a stable seam. Immediately after the fusion of this first capsule side and the resulting first seam (leading seam), the filling process with the capsule filling takes place. Immediately afterwards, the second seam (trailing seam) is closed.

The fusion process of the trailing seam in particular can easily be disrupted if the process conditions are not optimal, e.g., due to the effect of excessive restoring forces from the gel ribbons stretched by the capsule filling, or due to components of the filling getting between the gel ribbons. Such impairments to the seam formation, which can then lead to at least isolated cases of leaky seams (so-called leakers) between the capsule halves, are favored by factors such as high machine rotation speed or a lower thickness of the gel ribbons, which, on the other hand, may be desirable from an economic point of view.

The type of capsule filling can also have an influence on the quality of the seam formation, wherein surfactant, pasty, or particulate components of the filling, in particular the adjuvants contained therein, can get between the gel ribbons and become trapped in the seam.

Defective seams can lead to leaks immediately after production or even during further packaging and/or storage of the soft gelatin capsules. Even if only a few capsules are affected by this, it leads to a qualitative impairment of the entire batch.

### BRIEF SUMMARY OF THE INVENTION

The present invention relates to a soft gelatin capsule comprising a capsule shell and a filling encased by the capsule shell.

The invention also relates to a process for producing soft gelatin capsules and to the use of low-viscosity gelatin for producing soft gelatin capsules.

### BRIEF DESCRIPTION OF THE SEVERAL VIEWS OF THE DRAWING(S)

FIG. 1: photographic representation of the wall cross-section of a soft gelatin capsule according to the invention; and

FIG. 2: photographic representation of the wall cross-section of a soft gelatin capsule according to the prior art.

### DETAILED DESCRIPTION OF THE INVENTION

The object of the invention is that of proposing a soft gelatin capsule that can be produced with improved seam formation, thereby reducing the risk of leakers.

This object is achieved in the soft gelatin capsule of the type mentioned at the outset in accordance with the invention by the capsule shell comprising a low-viscosity gelatin having a viscosity of about 1.5 to about 2.4 mPa·s and a gel strength of about 150 to about 280 g Bloom, preferably of about 180 to about 250 g Bloom, in each case measured in a 6.67% gelatin solution at 60° C. according to the GME monograph “Standardised Methods for the Testing of Edible Gelatine”.

The term “low-viscosity gelatin” refers in the context of the present invention to the above-mentioned viscosity range of gelatin from about 1.5 to about 2.4 mPa·s, which is below the viscosity of typical gelatins for soft capsules according to the prior art (with viscosities of above about 2.5 to about 4 mPa·s). At the same time, the low-viscosity gelatin according to the invention exhibits typical gel strengths predominantly in the medium to high Bloom range.

The viscosity and gel strength characterize key physical and rheological properties of the gelatin and influence both the processability of the gelatin gel in the production process of the soft capsules and the properties of the produced capsule shell. These two aspects can result in conflicting requirements for the optimal properties of the gelatin to be used. Surprisingly, it was found in the course of the invention that a low-viscosity gelatin with the above-mentioned viscosity and Bloom values can be advantageously used in the production process for soft gelatin capsules and at the same time leads to soft gelatin capsules with improved seam formation. This advantage over standard gelatin can be demonstrated by the greater seam thickness and bursting force of the soft gelatin capsules produced.

While the reduced viscosity compared to typical gelatins improves the seam formation of the capsule shell, the viscosity in the range of about 1.5 to about 2.4 mPa·s (and with corresponding gel strength) is still sufficiently high to allow the gelatin gel to be processed without problems (such as the risk of ribbon breakage) during the production of the capsule shell using the rotary die process.

On the other hand, such low-viscosity gelatins or gelatin mixtures, which also have a low gel strength (typically well below 150 g Bloom) and which can be produced by selecting suitable parameters during the gelatin extraction process, are not suitable within the scope of the present invention, since they do not lead to a stable process during encapsulation using the rotary die process, nor to soft gelatin capsules with sufficient physical stability.

Surprisingly, it was also found that the soft capsules according to the invention require a significantly shorter drying time after encapsulation using the rotary die process than comparable soft capsules based on standard gelatin. The drying time required for the soft capsules according to the invention is in some cases shorter by a factor of about two, so that the production time can be significantly reduced by the invention.

The low-viscosity gelatin for the soft capsule according to the invention can be produced from known raw materials (typically skin or bones from cattle or pigs) using the acid hydrolysis process (type A gelatin) or the alkaline hydrolysis process (type B gelatin). Depending on the raw material and digestion process, the following viscosity and Bloom ranges are particularly preferred within the scope of the invention:
a type A gelatin obtained from pig skin, which has a viscosity of about 1.5 to about 2.3 mPa·s and a gel strength of about 150 to about 240 g Bloom, preferably of about 180 to about 220 g Bloom; or
a type A gelatin obtained from bovine bones, which has a viscosity of about 1.6 to about 2.4 mPa·s and a gel strength of about 160 to about 260 g Bloom, preferably of about 180 to about 230 g Bloom; or
a type B gelatin obtained from bovine bones, which has a viscosity of about 1.6 to about 2.4 mPa·s and a gel strength of about 160 to about 260 g Bloom, preferably of about 190 to about 240 g Bloom, more preferably of about 205 to about 240 g Bloom; or
a type B gelatin obtained from bovine skin, which has a viscosity of about 1.5 to about 2.3 mPa·s and a gel strength of about 160 to about 230 g Bloom, preferably of about 180 to about 220 g Bloom, more preferably of about 205 to about 220 g Bloom; or
a mixture of two or more of the aforementioned gelatins.

The low-viscosity gelatin is preferably produced by partial enzymatic hydrolysis of one or more starting gelatins which have a higher viscosity and a higher gel strength than the low-viscosity gelatin. Enzymatic treatment of gelatin with suitable proteases or peptidases shifts the molecular weight distribution of the protein chains contained in the starting gelatin, which regularly results in a reduction in viscosity and gel strength. By selecting the type and quantity of enzyme and the hydrolysis conditions (e.g., reaction time, temperature, and pH value), it is possible to specifically reduce the viscosity and gel strength of one or more starting gelatins to the above-mentioned target range for low-viscosity gelatin. When using several starting gelatins for enzymatic hydrolysis, these may be different gelatin extracts from the acidic or alkaline digestion of the collagen-containing raw material (such as skin or bones).

Partial enzymatic hydrolysis preferably comprises treatment with one or more endopeptidases. The enzymatic treatment of gelatin with endopeptidases has been known in the prior art for some time. Preferably, the endopeptidase is selected from collagenases, metalloproteases, serine proteases, cysteine proteases, pepsin, trypsin, chymotrypsin, and elastase.

According to an alternative embodiment of the invention, the low-viscosity gelatin is produced by microfiltration separation of higher molecular weight components from one or more starting gelatins, which have a higher viscosity and a higher gel strength than the low-viscosity gelatin.

The viscosity and, in particular, the gel strength of gelatin correlates to a certain extent with the average molecular weight. The low-viscosity gelatin according to the invention preferably has a weight-average molecular weight of about 45,000 to about 80,000 Da, wherein the molecular weight is determined in particular by gel permeation chromatography.

The low-viscosity gelatin preferably has a polydispersity of about 2.3 or less, more preferably of about 1.5 to about 2.3. As a quotient of the weight-average and number-average molecular weight (MW/MN), a low polydispersity correlates with a narrow molecular weight distribution of the protein chains in the gelatin. The polydispersity achievable with low-viscosity gelatin depends in particular on the raw material and the gelatin extraction process. Preferably, the low-viscosity gelatin comprises
a type A gelatin obtained from pig skin, which has a polydispersity of about 1.5 to about 1.9; or
a type A gelatin obtained from bovine bones, which has a polydispersity of about 1.8 to about 2.3; or
a type B gelatin obtained from bovine bones, which has a polydispersity of about 1.8 to about 2.3.

The corresponding starting gelatins from pig skin or bovine bones, from which these low-viscosity gelatins are produced, in particular by means of partial enzymatic hydrolysis or microfiltration, regularly have a higher polydispersity, typically a polydispersity of about 3 or more.

In the case of type B gelatin obtained from bovine skin, the low-viscosity gelatin preferably has a polydispersity of about 4 to about 6. Starting gelatins from type B bovine skin typically have a significantly higher polydispersity of about 8 or more.

With regard to the rheological properties of low-viscosity gelatin, it is also advantageous if the proportion of gelatin with a molecular weight of over 400,000 Da is as low as possible, and in particular less than about 1.5 wt. %. Gelatin fractions in this high molecular weight range are also referred to as microgel, which usually comprises cross-linked protein chains of alpha-collagen. According to the methods described above, this microgel can be degraded enzymatically or separated by microfiltration in order to produce the low-viscosity gelatin for the soft capsule according to the invention from one or more starting gelatins that have a higher viscosity and a higher gel strength.

In the soft gelatin capsule according to the invention, the capsule shell preferably comprises a proportion of about 50 to about 75 wt. % gelatin, more preferably about 55 to about 70 wt. %, in particular about 58 to about 67 wt. %. These figures relate to the dry matter of the gelatin, i.e., excluding the usual water content of gelatin of about 10 to 12 wt. %.

In a further advantageous embodiment of the invention, the capsule shell further comprises a collagen hydrolysate with a weight-average molecular weight of about 1,500 Da or less, determined by gel permeation chromatography. Collagen hydrolysate itself does not form a gel due to its low molecular weight, but such an addition can further improve the processability and seam formation of the soft gelatin capsule according to the invention. The capsule shell preferably comprises the collagen hydrolysate in a proportion of about 1 to about 8 wt. %.

Another advantage resulting from the addition of collagen hydrolysate is reduced cross-linking of the gelatin during storage of the soft gelatin capsule. Such cross-linking is undesirable because it negatively affects the dissolving behavior of the capsule shell.

Alternatively or in addition to collagen hydrolysate, the capsule shell may also advantageously comprise one or more free amino acids, preferably in a proportion of up to about 8 wt. %. The addition of amino acids, for example glycine, also counteracts the undesirable cross-linking of the gelatin.

Typically, the capsule shell also comprises one or more plasticizers, preferably in a proportion of about 20 to about 40 wt. %, more preferably from about 23 to about 34 wt. %, in relation to the dry matter of the plasticizer(s).

The plasticizer or plasticizers are preferably selected from sugar alcohols and non-reducing sugars, in particular glycerin, sorbitol, sorbitan, mannitol, maltitol, isomalt, isomaltulose, trehalose, and sucrose.

The capsule shell of the soft capsule according to the invention preferably has a water content of about 6 to about 14 wt. %.

The capsule shell may further comprise one or more colorants, preferably in a proportion of up to 6 wt. %.

The capsule shell may also comprise one or more flavorings, preferably in a proportion of up to 6 wt. %.

The capsule shell may further comprise one or more sweeteners, preferably in a proportion of up to 10 wt. %.

The capsule shell of the soft gelatin capsule according to the invention is preferably made from a gelatinous shell formulation comprising
about 35 to about 50 wt. % of low-viscosity gelatin, based on its dry matter;
about 15 to about 25 wt. % of one or more plasticizers, in relation to their dry matter;
about 25 to about 50 wt. % water;
optionally up to about 3 wt. % of one or more colorants;
optionally up to about 3 wt. % of one or more flavorings; and
optionally up to about 10 wt. % of one or more sweeteners.

In particular, the soft gelatin capsule is produced by means of the rotary die process from two material ribbons of the gelatinous shell formulation. It has been shown that such a shell formulation based on low-viscosity gelatin has excellent properties for this production process and at the same time results in a soft gelatin capsule with improved seam formation.

The capsule shell of the soft gelatin capsule according to the invention preferably has a wall thickness of about 100 to about 600 μm, more preferably of about 150 to about 550 μm, in particular of about 200 to about 500 μm. The wall thickness of the capsule shell can be influenced by the composition of the gelatinous shell formulation and/or by the process parameters.

The filling encased by the capsule shell can be in the form of a solution, a suspension, an emulsion, or a paste-like filling in the soft gelatin capsule according to the invention. The filling can be either hydrophilic, i.e., comprise a polyethylene glycol-based solution or suspension, or lipophilic or hydrophobic, i.e., comprise an oil or an oily emulsion.

The filling of the soft gelatin capsule according to the invention may comprise one or more pharmaceutical active substances for administration as a drug, or one or more nutraceutical ingredients for administration as a dietary supplement. In addition to the active substances or the ingredients, the filling typically comprises one or more adjuvants.

A further object of the invention is that of proposing a process for producing soft gelatin capsules with improved seam formation.

This object is achieved in accordance with the invention by a process comprising the steps of:
a) providing a gelatinous shell formulation comprising
   about 35 to about 50 wt. % of a low-viscosity gelatin, in relation to its dry matter, which has a viscosity of about 1.5 to about 2.4 mPa·s and a gel strength of about 150 to about 280 g Bloom, preferably of about 180 to about 250 g Bloom, in each case measured in a 6.67% gelatin solution at 60° C. according to the GME monograph “Standardised Methods for the Testing of Edible Gelatin”;
   about 15 to about 25 wt. % of one or more plasticizers, in relation to their dry matter;
   about 25 to about 50 wt. % water;
   optionally up to about 3 wt. % of one or more colorants;
   optionally up to about 3 wt. % of one or more flavorings; and
   optionally up to about 10 wt. % of one or more sweeteners,
b) providing a capsule filling, and
c) producing the soft gelatin capsules by encasing the capsule filling with a capsule shell formed from the shell formulation.

The soft gelatin capsules are preferably produced in step c) using the rotary die process from two material ribbons of the gelatinous shell formulation.

Particular advantages and further preferred embodiments of the process according to the invention have already been explained in conjunction with the soft gelatin capsule according to the invention.

A further aspect of the invention relates to the use of a low-viscosity gelatin for producing soft gelatin capsules, wherein the gelatin has a viscosity of about 1.5 to about 2.4 mPa·s and a gel strength of about 150 to about 280 g Bloom, preferably of about 180 to about 250 g Bloom, in each case measured in a 6.67% gelatin solution at 60° C. according to the GME monograph “Standardised Methods for the Testing of Edible Gelatin.”

These and other advantages of the invention are explained in more detail in the following exemplary embodiments, which do not limit the scope of protection in any way.

The following examples further illustrate the invention but, of course, should not be construed as in any way limiting its scope.

### Example 1

### Production of Low-Viscosity Gelatins

The production of a low-viscosity gelatin for the capsule shell of a soft gelatin capsule according to the invention by means of partial enzymatic hydrolysis can be carried out, for example, according to the following protocols:

1.1 Gelatin Type B from Bovine Bones

A commercially available gelatin (type B from bovine bones) with a standard viscosity of 5.5 mPa·s and a gel strength of 288 g Bloom is used as the starting gelatin. 50 liters of a 20% w/w aqueous solution of this gelatin (initial weight 10 kg, dry matter 18%) are heated to 55° C. with moderate stirring (fast, but without introducing air) and the pH is adjusted to 5.7. The kinematic starting viscosity of this gelatin solution at 55° C. is 140 mPa·s.

A total of 4.8 ml of a 40% solution of the enzyme Bacillus lolysin (840 UHb/g), a bacterial metalloprotease (endopeptidase), is added in three steps of 1.6 ml each during the first 20 minutes. After one hour, the kinematic viscosity of the solution is 21.7 mPa·s, which corresponds to the desired standard viscosity of the partially hydrolyzed gelatin.

The protease is deactivated by briefly heating to 80° C., the solution is gelled by cooling, and the gelatin is then dried. The standard viscosity of the gelatin obtained (in a 6.67% solution at 60° C.) is 2.1 mPa·s and the gel strength is 236 g Bloom.

1.2 Type A Gelatin from Pig Skin

Commercially available gelatin (type A from pig skin) with a standard viscosity of 3.2 mPa·s and a gel strength of 300 g Bloom is used as the starting gelatin. 25 liters of a 30% w/w aqueous solution of this gelatin (initial weight 7.5 kg, dry matter 27.5%) are heated to 55° C. with moderate stirring (fast, but without introducing air) and the pH is adjusted to 5.7. The kinematic starting viscosity of this gelatin solution at 55° C. is 530 mPa·s.

After adding 410 μl of an enzyme solution (endopeptidase COROLASE®7089) and a reaction time of 115 min, the kinematic viscosity of the solution is about 82 mPa·s, corresponding to a standard viscosity of the gelatin contained (in a 6.67% solution at 60° C.) of 1.8 mPa·s. The gel strength of the gelatin produced is 191 g Bloom.

### 1.3 to 1.9 Other Low-Viscosity Gelatins

In general, this method of partial enzymatic hydrolysis enables the production of low-viscosity gelatins from different types of starting gelatins with moderate Bloom loss. To determine the reaction time required for a given amount of a specific enzyme, the kinematic viscosity of the gelatin solution can be measured during enzymatic hydrolysis, as described in the above example, until it corresponds to the desired standard viscosity of the gelatin, i.e. a viscosity of about 1.5 to about 2.4 mPa·s. Based on these results, the amount of enzyme can also be varied and, if necessary, adjusted to an optimal value in order to achieve the target viscosity of the gelatin in a specified reaction time of, for example, 30 min.

Parallel to the reduction in viscosity through partial enzymatic hydrolysis, there is also a reduction in gel strength, wherein it has been shown that the desired gel strength of the low-viscosity gelatin in the range of about 150 to about 280 g Bloom can also be easily achieved using the procedure described.

Table 1 below shows the start and end values of the standard viscosity and gel strength for Examples 1.1 and 1.2 above, as well as for a number of other low-viscosity gelatins that can be obtained by partial enzymatic hydrolysis. The gelatin type, weight-average molecular weight (MW), and polydispersity (MW/MN) are also given. The type B gelatins are each obtained from bovine bones, while the type A gelatins are obtained from pig skin (Example 1.2) or bovine bones (Example 1.3).

TABLE 1

| | | Start | End | Start | End | | |
|---|---|---|---|---|---|---|---|
| | | viscos. | viscos. | Bloom | Bloom | MW | MW/ |
| Ex. | Type | [m · Pas] | [m · Pas] | [g] | [g] | [Da] | MN |
| 1.1 | B | 5.5 | 2.1 | 288 | 236 | 59.753 | 1.8 |
| 1.2 | A | 3.2 | 1.8 | 300 | 191 | 50.371 | 1.6 |
| 1.3 | A | 2.9 | 1.7 | 282 | 164 | 54.047 | 2.2 |
| 1.4 | B | 5.2 | 2.0 | 315 | 238 | 56.544 | 1.8 |
| 1.5 | B | 6.4 | 2.1 | 270 | 194 | 68.261 | 2.1 |
| 1.6 | B | 8.1 | 2.1 | 244 | 163 | 67.340 | 2.1 |
| 1.7 | B | 7.5 | 2.2 | 275 | 263 | 75.037 | 2.1 |
| 1.8 | B | 6.1 | 2.3 | 268 | 237 | 75.375 | 2.1 |
| 1.9 | B | 7.9 | 2.4 | 214 | 148 | 79.500 | 2.3 |

The weight-average molecular weight of the gelatins is determined by gel permeation chromatography using a TSK 4000 SWXL column (Tosoh Bioscience GmbH) and a phosphate buffer with 1% SDS at pH 5.3. A reference gelatin is used for calibration. The protein content in the eluent is measured using a UV detector at a wavelength of 210 nm.

In contrast to the enzymatically treated gelatins in Table 1, other low-viscosity gelatins that also have a low gel strength (typically well below 150 g Bloom) and can be produced by selecting suitable parameters during the gelatin extraction process are not suitable for the production of soft gelatin capsules according to the invention.

### Example 2

### Composition of the Capsule Shell

The soft gelatin capsules according to the invention are produced from a gelatinous shell formulation which, in addition to the low-viscosity gelatin, substantially comprises water and preferably one or more plasticizers.

Exemplary compositions of the capsule shell resulting from shell formulations with different plasticizers are given in Table 2 below. These are therefore the quantitative composition after the drying process, in which the water content of the shell formulation is reduced from about 25 to about 50 wt. % to the water content of the capsule shell of about 6 to about 10 wt. %. All figures are given in weight percent and refer to the dry mass of the gelatin and the plasticizers.

TABLE 2

| | | | | | Sorbitol | |
|---|---|---|---|---|---|---|
| | | | | | + | |
| Ex. | Gelatin | Glycerin | Sorbitol | Sucrose | Sorbitan | Water |
| 2.1 | 60.0-62.7 | 30.0-31.3 | — | — | — | 6.0-10.0 |
| 2.2 | 60.0-62.7 | 15.0-15.7 | — | — | 15.0- | 6.0-10.0 |
| | | | | | 15.7 | |
| 2.3 | 60.0-62.7 | 7.50-7.83 | 22.5-23.5 | — | — | 6.0-10.0 |
| 2.4 | 60.0-62.7 | 7.50-7.83 | — | 7.50-7.83 | — | 6.0-10.0 |
| 2.5 | 60.0-62.7 | — | — | 30.0-31.3 | — | 6.0-10.0 |
| 2.6 | 64.3-67.1 | 25.7-26.9 | — | — | — | 6.0-10.0 |
| 2.7 | 64.3-67.1 | 12.9-13.4 | — | — | 12.9- | 6.0-10.0 |
| | | | | | 13.4 | |
| 2.8 | 64.3-67.1 | 19.3-20.1 | 6.43-6.71 | — | — | 6.0-10.0 |
| 2.9 | 64.3-67.1 | — | — | 25.7-26.9 | — | 6.0-10.0 |
| 2.10 | 64.3-67.1 | 6.43-6.71 | — | 19.3-20.1 | — | 6.0-10.0 |
| 2.11 | 58.1-60.7 | 31.9-33.4 | — | — | — | 6.0-10.0 |
| 2.12 | 58.1-60.7 | 16.0-16.7 | — | — | 16.0- | 6.0-10.0 |
| | | | | | 16.7 | |
| 2.13 | 58.1-60.7 | 7.98-8.34 | 24.0-25.0 | — | — | 6.0-10.0 |
| 2.14 | 58.1-60.7 | 24.0-25.0 | — | 7.98-8.34 | — | 6.0-10.0 |
| 2.15 | 58.1-60.7 | — | — | 31.9-33.4 | — | 6.0-10.0 |

### Example 3

### Production of Soft Gelatin Capsules on a Laboratory Scale

The soft gelatin capsules according to the invention are typically produced using the known rotary die process. To investigate the seam formation properties, various soft capsules were produced in this way from shell formulations on a laboratory scale, which were based on the low-viscosity gelatins according to Examples 1.1, 1.2, or 1.3, and on the other hand on standard soft capsule gelatins (a type B bovine bone gelatin with a viscosity of 3.3 mPa·s and 159 g Bloom or a type A pig skin gelatin with a viscosity of 2.7 mPa·s and 198 g Bloom).

Glycerin and a mixture of sorbitol and sorbitan (ratio 60:40) were used as plasticizers in equal parts, wherein the composition of the capsule shell after drying corresponds to Example 2.7 above.

During the production of the capsules from these shell formulations according to the standardized procedure, the machine rotation speeds (2.0 rpm and 3.5 rpm) and the temperature of the filling wedge between 38.5° C. and 43.5° C. were also varied. After changing the rotation speed, the thickness of the gel ribbon is readjusted to 700 μm by opening the spreader box gap.

After complete drying in the tumble and/or rack dryer, the capsules were examined for seam thickness and bursting force.

### Example 4

### Measurement of Seam Thicknesses and Bursting Force

When producing soft gelatin capsules from two gel ribbons, as described at the outset, a leading seam is first formed and then, after the capsule filling has been inserted, a trailing seam. Due to the principle involved, the trailing seam is always the weaker of the two seams, so the thickness of the trailing seam was measured and compared as a key parameter for the seam formation properties of the gelatin.

To measure the seam thickness, the dried capsules are divided into two about equal halves at their most bulging radius perpendicular to the seams, emptied and cleaned, and then measured under a magnifying lens. In addition to the absolute seam thickness, the relative seam thickness is also measured, i.e. the ratio of the wall thickness directly at the seam to the maximum wall thickness next to the seam, where material accumulates during seam formation. The relative seam thickness is even more significant than the absolute seam thickness for seam formation properties, as it is less dependent on the process conditions.

The bursting force of the soft capsules is measured as a further parameter for the seam formation properties. The bursting force is the force required to burst the soft capsule, which is arranged between two pressing tools in such a way that its seam lies parallel to the surface of the force-applying tool jaws. The bursting force can be measured, for example, with the “Texture Analyzer TA-XTplus” testing device or the “Zwick/Roell Z2.5/TN1S” testing device.

The absolute and relative seam thicknesses and bursting forces for the soft gelatin capsules produced from the shell formulations described above are given in Table 3 for machine rotation speeds of 2.0 rpm and in Table 4 for machine rotation speeds of 3.5 rpm.

Other low-viscosity gelatins, which also have a low gel strength (typically well below 150 g Bloom) and which can be produced by selecting suitable parameters during the gelatin extraction process, do not result in a stable process in the event of encapsulation using the rotary die method, nor do they produce gelatin soft capsules with sufficient physical stability.

TABLE 3
Results at 2.0 rpm

| | Wedge | Thickness | | Relative | Bursting | |
|---|---|---|---|---|---|---|
| | Temp. | trailing | | seam | force | |
| Gelatin | [° C.] | seam [μm] | SD | thickness | [kg] | SD |
| Standard | 40.5 | 272 | 17.5 | 70% | 32.6 | 7.0 |
| Type B | | | | | | |
| Standard | 40.5 | 307 | 11.3 | 74% | 22.0 | 4.1 |
| Type A | | | | | | |
| Ex. 1.1 | 40.5 | 373 | 25.9 | 76% | 46.5 | 7.3 |
| Ex. 1.1 | 41.5 | 339 | 27.6 | 72% | 43.9 | 5.6 |
| Ex. 1.2 | 40.5 | 403 | 11.0 | 78% | 43.7 | 7.3 |
| Ex. 1.3 | 42.5 | 401 | 33.0 | 69% | 50.5 | 4.5 |

TABLE 4
Results at 3.5 rpm

| | Wedge | Thickness | | Relative | Bursting | |
|---|---|---|---|---|---|---|
| | Temp. | trailing | | seam | force | |
| Gelatin | [° C.] | seam [μm] | SD | thickness | [kg] | SD |
| Standard | 39.5 | 142 | 12 | 34% | 15.2 | 7.0 |
| Type B | | | | | | |
| Standard | 40.5 | 164 | 19 | 41% | 13.6 | 1.4 |
| Type A | | | | | | |
| Ex. 1.1 | 43.5 | 231 | 23 | 47% | 33.0 | 2.6 |
| Ex. 1.1 | 43.5 | 233 | 25 | 61% | 22.6 | 1.7 |
| Ex. 1.2 | 40.5 | 275 | 20 | 58% | 28.2 | 8.9 |
| Ex. 1.3 | 38.5 | 243 | 25 | 53% | 30.0 | 3.1 |

The results show that for both machine rotation speeds, the absolute seam thickness of the trailing seam and the bursting force are significantly higher for all soft capsules according to the invention than for soft capsules made from standard gelatins. At the higher speed of 3.5 rpm, this also applies to the relative seam thickness.

Increasing the machine rotation speed generally leads to a decrease in absolute seam thickness, as can also be seen in the comparison.

The correlation between the seam thickness and the bursting force also shows that the trailing seam is relevant as a potential weak point for the mechanical properties of the capsule shell and thus represents the main cause of leaky capsules (so-called leakers).

By using a low-viscosity gelatin according to the present invention, the seam formation properties of soft gelatin capsules can be significantly improved, thereby reducing the risk of leakers.

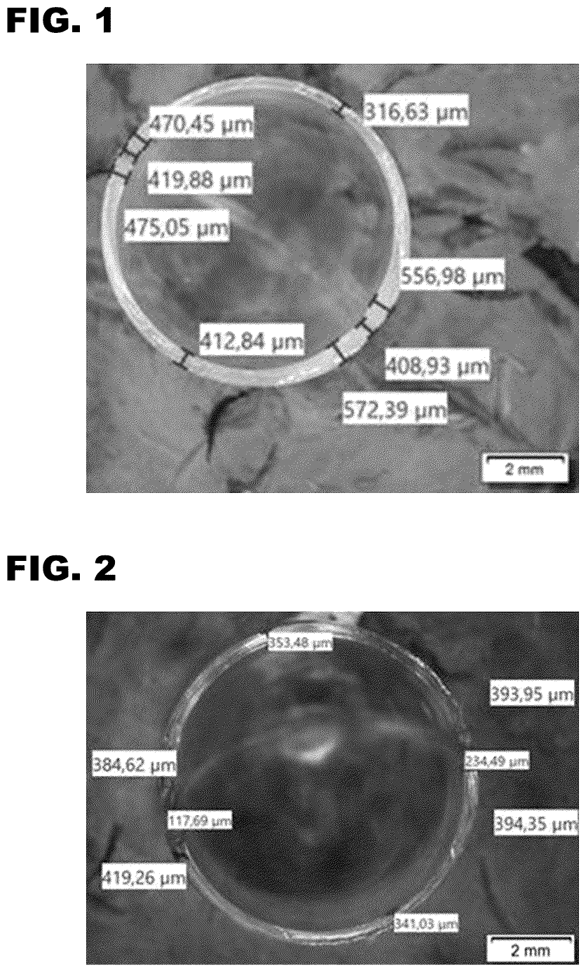

FIG. 1 shows a photographic representation of the wall cross-section of a soft gelatin capsule according to the invention, which can be used to measure the absolute and relative seam thicknesses as described above. In this example, the seam is located at the bottom right of the image and has an absolute thickness of 408.93 μm. The maximum wall thickness next to the seam, where material accumulation occurs during seam formation, is 572.39 μm, so that the relative thickness of the seam is 71%.

FIG. 2 shows a corresponding photographic representation of the wall cross-section of a soft gelatin capsule according to the prior art, i.e., based on a standard capsule gelatin. The trailing seam is located on the left side and has an absolute thickness of 117.69 μm, which is a relative thickness of 28% in relation to the adjacent maximum wall thickness of 419.26 μm.

A comparison of FIGS. 1 and 2 also shows that, in the soft capsule according to the invention, the material enrichment in the seam area is significantly more pronounced overall than in the capsule according to the prior art, i.e. the absolute seam thickness in relation to the average wall thickness of the capsule (or to the wall thickness between the two seams) is significantly increased. In other words, and as can be clearly seen in the Figures, the seam in FIG. 2 (prior art) is significantly thinner than the average wall thickness, while in FIG. 1 (capsule according to the invention) it is equal to or even greater than the average wall thickness.

### Example 5

### Production of Soft Gelatin Capsules on an Industrial Scale

To investigate the seam formation properties, soft capsules with different fillings were also produced on an industrial-scale rotary die machine (production quantity >100,000 capsules). The shell formulations in these examples include the low-viscosity gelatin according to Example 1.1 above and, for comparison, a standard soft capsule gelatin, namely type B bovine bone gelatin with a viscosity of 3.3 mPa·s and 159 g Bloom.

Glycerin was used as a plasticizer, with the composition of the capsule shell after drying corresponding to Example 2.6 above.

Table 5 shows the absolute seam thicknesses, bursting forces, and other process parameters for the soft gelatin capsules produced from the shell formulations described above with different fillings (krill oil, sunflower oil, and polysorbate). The seam thicknesses and bursting forces were measured as described in Example 4.

TABLE 5
Results with different fillings

| | | | | Thickness | Thickness | | | |
|---|---|---|---|---|---|---|---|---|
| | | Wedge | Rotation | of gel | of trailing | | Bursting | |
| | | temp | speed | ribbon | seam | | force | |
| Filling | Gelatin | [° C.] | [rpm] | [μm] | [μm] | SD | [kg] | SD |
| Krill oil | Standard | 44 | 2.7 | 720 | 83 | 9 | 6.8 | 0.8 |
| | type B | | | | | | | |
| | Ex. 1.1 | 42 | 2.7 | 720 | 173 | 39 | 13.6 | 0.8 |
| Suspension | Standard | 45 | 2.8 | 850 | 176 | 31 | 9.0 | 2.5 |
| of | type B | | | | | | | |
| blueberry | Ex. 1.1 | 44 | 2.8 | 820 | 250 | 36 | 10.1 | 2.2 |
| extract in | | | | | | | | |
| sunflower | | | | | | | | |
| oil | | | | | | | | |
| Polysorbate | Standard | 44 | 3.0 | 850 | 207 | 33 | 8.5 | 2.1 |
| | type B | | | | | | | |
| | Ex. 1.1 | 39 | 3.0 | 850 | 314 | 31 | 15.0 | 2.0 |

These tests also show a significant increase in absolute seam thickness when using low-viscosity gelatin compared to standard soft capsule gelatin, depending on the filling, by about 108%, about 42%, and about 51%, respectively. At the same time, the burst force increased by about 100%, about 12%, and about 76%, respectively.

In a further series of tests, the influence of the rotation speed on the thickness of the trailing seam was investigated. The low-viscosity gelatin according to Example 1.1 was used in comparison with a standard soft capsule gelatin (bovine bone gelatin type B with a viscosity of 3.3 mPa·s and 159 g Bloom).

Glycerin was used as a plasticizer, with the composition of the capsule shell after drying corresponding to Example 2.6 above. A suspension of blueberry extract in sunflower oil (dry matter 25%) was used as the filling.

The absolute seam thicknesses of the soft capsules produced and other process parameters are given in Table 6.

TABLE 6
Results with different rotational speeds

| | Wedge | Rotational | Thickness of | Thickness of | |
|---|---|---|---|---|---|
| | temp | speed | gel ribbon | trailing seam | |
| Gelatin | [° C.] | [rpm] | [μm] | [μm] | SD |
| Standard | 43 | 2.7 | 720 | 107 | 24 |
| Type B | | | | | |
| Ex. 1.1 | 44 | 2.7 | 720 | 177 | 22 |
| Ex. 1.1 | 44 | 2.9 | 715 | 164 | 23 |
| Ex. 1.1 | 44 | 3.1 | 700 | 143 | 26 |
| Ex. 1.1 | 44 | 3.3 | 680 | 163 | 24 |
| Ex. 1.1 | 44 | 3.6 | 630 | 103 | 18 |

When using low-viscosity gelatin, rotation speeds of 2.7 to 3.3 rpm can be used to achieve seam thicknesses that are between about 34% and about 65% higher than the seam thickness achieved with standard gelatin at 2.7 rpm. Only when the speed is further increased to 3.6 rpm does the seam thickness fall back to about the level of standard gelatin at 2.7 rpm.

In the case of soft capsules with standard gelatin, all capsules in a batch showed leakers after drying in the tumble dryer. By contrast, between 70% and 89% of the soft capsules with low-viscosity gelatin in a batch were free of leaks. This also demonstrates the significantly improved seam formation in the soft capsules according to the invention.

The drying time required for the soft capsules was 38 hours when standard gelatin was used (final capsule hardness 10.0 N), but only 18 hours when low-viscosity gelatin was used (final capsule hardness 10.6 N). The soft capsules according to the invention thus require a significantly shorter drying time than soft capsules made from standard gelatin, in this example less than half the time.

1. A soft gelatin capsule comprising a capsule shell and a filling encased by the capsule shell,
wherein the capsule shell comprises a low-viscosity gelatin having a viscosity of about 1.5 to about 2.4 mPa·s and a gel strength of about 150 to about 280 g Bloom, in each case measured in a 6.67% gelatin solution at 60° C. according to the GME monograph “Standardised Methods for the Testing of Edible Gelatin.”

2. The soft gelatin capsule in accordance with claim 1, wherein the low-viscosity gelatin comprises
a type A gelatin obtained from pig skin, which has a viscosity of about 1.5 to about 2.3 mPa·s and a gel strength of about 150 to about 240 g Bloom; or
a type A gelatin obtained from bovine bones, which has a viscosity of about 1.6 to about 2.4 mPa·s and a gel strength of about 160 to about 260 g Bloom; or
a type B gelatin obtained from bovine bones, which has a viscosity of about 1.6 to about 2.4 mPa·s and a gel strength of about 160 to about 260 g Bloom; or
a type B gelatin obtained from bovine skin, which has a viscosity of about 1.5 to about 2.3 mPa·s and a gel strength of about 160 to about 230 g Bloom; or
a mixture of two or more of the aforementioned gelatins.

3. The soft gelatin capsule in accordance with claim 1, wherein the low-viscosity gelatin is produced by partial enzymatic hydrolysis of one or more starting gelatins which have a higher viscosity and a higher gel strength than the low-viscosity gelatin.

4. The soft gelatin capsule in accordance with claim 3, wherein the partial enzymatic hydrolysis comprises treatment with one or more endopeptidases selected from collagenases, metalloproteases, serine proteases, cysteine proteases, pepsin, trypsin, chymotrypsin, and elastase.

5. The soft gelatin capsule in accordance with claim 1, wherein the low-viscosity gelatin is produced by microfiltration separation of higher molecular weight components from one or more starting gelatins that have a higher viscosity and a higher gel strength than the low-viscosity gelatin.

6. The soft gelatin capsule in accordance with claim 1, wherein the low-viscosity gelatin has a weight-average molecular weight of about 45,000 to about 80,000 Da, determined by gel permeation chromatography.

7. The soft gelatin capsule in accordance with claim 1, wherein the low-viscosity gelatin has a polydispersity of about 2.3 or less, and/or wherein the low-viscosity gelatin comprises
a type A gelatin obtained from pig skin, which has a polydispersity of about 1.5 to about 1.9; or
a type A gelatin obtained from bovine bones, which has a polydispersity of about 1.8 to about 2.3; or
a type B gelatin obtained from bovine bones, which has a polydispersity of about 1.8 to about 2.3.

8. The soft gelatin capsule in accordance with claim 1, wherein the low-viscosity gelatin comprises a type B gelatin obtained from bovine skin, which has a polydispersity of about 4 to about 6.

9. The soft gelatin capsule in accordance with claim 1, wherein the low molecular weight gelatin has a proportion of gelatin with a molecular weight of over 400,000 Da that is less than about 1.5 wt. %.

10. Ti soft gelatin capsule in accordance with claim 1, wherein the capsule shell comprises a proportion of about 50 to about 75 wt. % gelatin, in relation to the dry matter of the gelatin.

11. The soft gelatin capsule in accordance with claim 1, wherein the capsule shell further comprises a collagen hydrolysate with a weight-average molecular weight of about 1,500 Da or less, determined by gel permeation chromatography, in a proportion of about 1 to about 8 wt. %.

12. The soft gelatin capsule in accordance with claim 1, wherein the capsule shell further comprises one or more free amino acids in a proportion of up to about 8 wt. %.

13. The soft gelatin capsule in accordance with claim 1, wherein the capsule shell further comprises one or more plasticizers in a proportion of about 20 to about 40 wt. %, in relation to the dry matter of the plasticizer(s).

14. The soft gelatin capsule in accordance with claim 13, wherein the plasticizer or plasticizers are selected from sugar alcohols and non-reducing sugars, comprising glycerin, sorbitol, sorbitan, mannitol, maltitol, isomalt, isomaltulose, trehalose, and sucrose.

15. The soft gelatin capsule in accordance with claim 1, wherein the capsule shell has a water content of about 6 to about 14 wt. %.

16. The soft gelatin capsule in accordance with the claim 1, wherein the capsule shell further comprises one or more colorants in a proportion of up to about 6 wt. %.

17. The soft gelatin capsule in accordance with claim 1, wherein the capsule shell further comprises one or more flavorings in a proportion of up to about 6 wt. %.

18. The soft gelatin capsule in accordance with claim 1, wherein the capsule shell further comprises one or more sweeteners-preferably in a proportion of up to about 10 wt. %.

19. The soft gelatin capsule in accordance with claim 1, wherein the capsule shell is made from a gelatinous shell formulation comprising
about 35 to about 50 wt. % of low-viscosity gelatin, in relation to its dry matter;
about 15 to about 25 wt. % of one or more plasticizers, in relation to their dry matter;
about 25 to about 50 wt. % water;
optionally up to about 3 wt. % of one or more colorants;
optionally up to about 3 wt. % of one or more flavorings; and
optionally up to about 10 wt. % of one or more sweeteners.

20. The soft gelatin capsule in accordance with claim 19, wherein the soft gelatin capsule is produced by means of the rotary die process from two material ribbons of the gelatinous shell formulation.

21. The soft gelatin capsule in accordance with claim 1, wherein the capsule shell has a wall thickness of about 100 to about 600 μm.

22. The soft gelatin capsule in accordance with claim 1, wherein the filling encased by the capsule shell is in the form of a solution, a suspension, an emulsion, or a paste-like filling.

23. The soft gelatin capsule in accordance with claim 1, wherein the filling is hydrophilic or lipophilic.

24. The soft gelatin capsule in accordance with claim 1, wherein the filling comprises one or more pharmaceutical or nutraceutical ingredients and one or more adjuvants.

25. A process for producing soft gelatin capsules, comprising the steps of:
a) providing a gelatinous shell formulation comprising
about 35 to about 50 wt. % of a low-viscosity gelatin, in relation to its dry matter, which has a viscosity of about 1.5 to about 2.4 mPa·s and a gel strength of about 150 to about 280 g Bloom, in each case measured in a 6.67% gelatin solution at 60° C. according to the GME monograph “Standardised Methods for the Testing of Edible Gelatin”;
about 15 to about 25 wt. % of one or more plasticizers, in relation to their dry matter;
about 25 to about 50 wt. % water;
optionally up to about 3 wt. % of one or more colorants;
optionally up to about 3 wt. % of one or more flavorings; and
optionally up to about 10 wt. % of one or more sweeteners,
b) providing a capsule filling, and
c) producing the soft gelatin capsules by encasing the capsule filling with a capsule shell formed from the shell formulation.

26. (canceled)
